(19) United States
(12) Patent Application Publication
(10) Pub. No.: US 2019/0321255 A1
(43) Pub. Date: Oct. 24, 2019

(54) CPR ASSIST DEVICE

(71) Applicant: HENG-SHIUAN JANG, Kaohsiung City (TW)

(72) Inventor: HENG-SHIUAN JANG, Kaohsiung City (TW)

(21) Appl. No.: 15/959,227

(22) Filed: Apr. 22, 2018

(51) Int. Cl. A61H 31/00; A61N 1/39

(52) U.S. Cl. CPC A61H 31/005; A61N 1/3993; A61H 2201/5071; A61N 1/3925; A61H 2201/5043

(57) ABSTRACT

A CPR assist device primarily comprises a wearable device, an AED, and an oxygen supplying device, with all the devices stored in a case body. On the wearable device are disposed a covering member and straps connecting to the covering member. On the covering member are disposed a pressure detecting unit, a heartbeat detecting unit, a first display unit and a second display unit. In case of a cardiac arrest, the covering member is put on the chest of the patient for the rescuer to administer CPR at a particular position. During CPR administration the pressure detecting unit detects the compression force and displays it on the first display unit to inform the rescuer as to whether the compressions are being administered properly. Further, the heartbeat detecting unit detects the heartbeat signals, and the second display unit shows whether the heart has started beating again to facilitate proper CPR administration. The CPR success rate may be further increased with the aid of the AED and oxygen supplying device.

### BACKGROUND OF THE INVENTION

### 1. Field of the Invention

The present invention relates to a CPR assist device and particularly to a CPR assist device which may inform the rescuer as to whether chest compressions are being administered properly so that the CPR success rate may be increased.

### 2. Description of Related Art

There are in average around 20,000 cases of sudden cardiac arrest occurring per year in Taiwan. Since the golden time in CPR is 5 minutes, early CPR increases the survival rate. Statistics show that in Taiwan the survival rate of patients who receive CPR 5 minutes before hospital arrival is around 15-20%. However, although the general public in Taiwan has basic ideas about CPR, its proper procedures are not well-known.

In conventional CPR the rescuer administers artificial ventilation and chest compressions on the patient to help the patient maintain blood oxygenation. However, manual chest compressions vary in depth and rate, which often reduces the effect of CPR. Further, regarding the depth of chest compressions, due to a current lack in any available assist device the rescuer has only his judgment to rely on. In view of this, after laborious research and experiments, the applicant has devised a CPR assist device which may inform the rescuer as to whether chest compressions are being administered properly so that the CPR success rate may be increased.

### SUMMARY OF THE INVENTION

It is a primary objective of the present invention to provide a CPR assist device which may inform the rescuer as to whether chest compressions are being administered properly so that the CPR success rate may be increased.

A foregoing CPR assist device comprises a wearable device, an AED, and an oxygen supplying device, with all the devices stored in a case body. On the wearable device are disposed a covering member and straps connecting to the covering member. On the covering member are disposed a pressure detecting unit, a heartbeat detecting unit, a first display unit and a second display unit. In case of a cardiac arrest, the covering member is put on the chest of the patient for the rescuer to administer CPR at a particular position. During CPR administration the pressure detecting unit detects the compression force and displays it on the first display unit to inform the rescuer as to whether the compressions are being administered properly. Further, the heartbeat detecting unit detects the heartbeat signals, and the second display unit shows whether the heart has started beating again to facilitate proper CPR administration. The CPR success rate may be further increased with the aid of the AED and oxygen supplying device.

A foregoing CPR assist device, wherein the first display unit and the second display unit disposed on the covering member are light-emitting diodes. The compression force and heartbeat signals are displayed by the blinking frequency of the LED.

A foregoing CPR assist device, wherein the oxygen supplying device includes an oxygen bottle and a mask connecting thereto for covering the mouth and nose of the patient.

### BRIEF DESCRIPTION OF THE DRAWINGS

FIG. 1 is a three-dimensional view of the present invention;

FIG. 2 is a circuit diagram of a wearable device according to a preferred embodiment of the present invention;

FIG. 3 and FIG. 4 are drawings of the present invention in use;

FIG. 5 is a drawing of the present invention providing oxygen; and

FIG. 6 is a drawing of an AED in use according to the present invention.

### DETAILED DESCRIPTION OF THE PREFERRED EMBODIMENTS

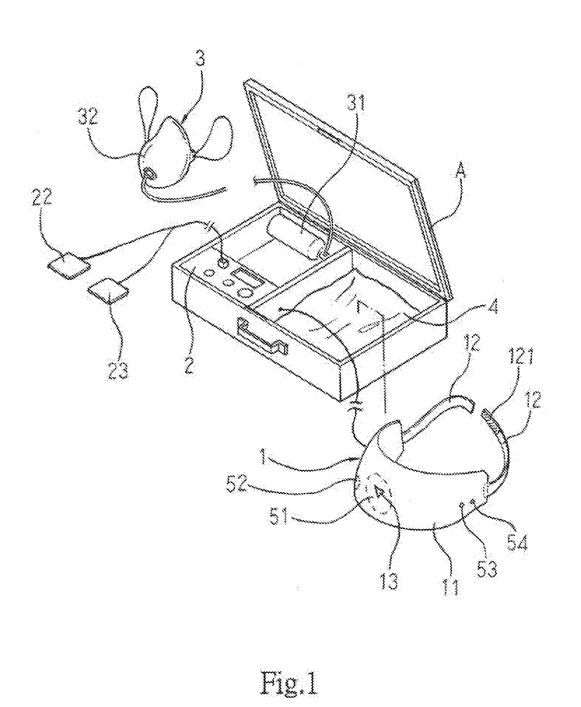

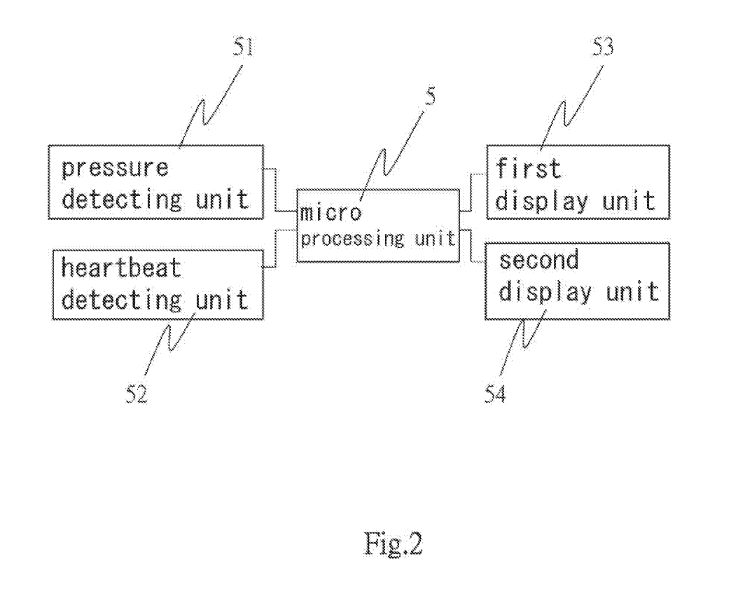

Please refer to both FIG. 1 and FIG. 2, showing respectively a three-dimensional view of the present invention and a circuit diagram of a wearable device according to a preferred embodiment of the present invention. As the figures show, the present invention comprises a wearable device 1, an AED 2, and an oxygen supplying device 3, with all the devices stored in a case body A. On the wearable device 1 are disposed a covering member 11 and two straps 12 connecting to the covering member 11. In the present embodiment, at the tail ends of the two straps 12 are disposed sticky buckles 121 for the two to connect with each other. On the covering member 11 is disposed a micro processing unit 5, whereto are connected a pressure detecting unit 51, a heartbeat detecting unit 52, a first display unit 53 and a second display unit 54. In the present embodiment, the first display unit 53 and the second display unit 54 are light-emitting diodes.

The pressure detecting unit 41 of the present invention is disposed at a correct position for administering CPR (at the notch in the center of the chest where the breastbone meets the ribs, that is, at the central point of the line connecting the two nipples). Further, on the surface of the covering member 11 is disposed a mark 13 to facilitate the rescuer's identification of the correct position for administering CPR.

A plurality of control buttons 21 are disposed on the AED 2, thereinto may be plugged an electrode pad 22 and an electrode pad 23.

The oxygen supplying device 3 comprises an oxygen bottle 31 and a mask 32 connecting thereto for covering the mouth and nose of the patient.

A combination of the foregoing elements forms a CPR assist device. In case of a sudden cardiac arrest, take out the head rest 4, place it on the floor or where the patient may lie flat. Place the head of the patient on the head rest 4 after the patient lies flat. Then put the covering member 11 on the chest of the patient for the rescuer to administer CPR at a particular position. During CPR administration the pressure detecting unit 51 detects the compression force, which is displayed on the first display unit 53 to inform the rescuer as to whether the compressions are being administered properly. Further, the heartbeat detecting unit 52 detects the heartbeat signals, and the second display unit 54 shows whether the heart has started beating again to facilitate proper CPR administration. The CPR success rate may be further increased with the aid of the AED 2 and oxygen supplying device 3.

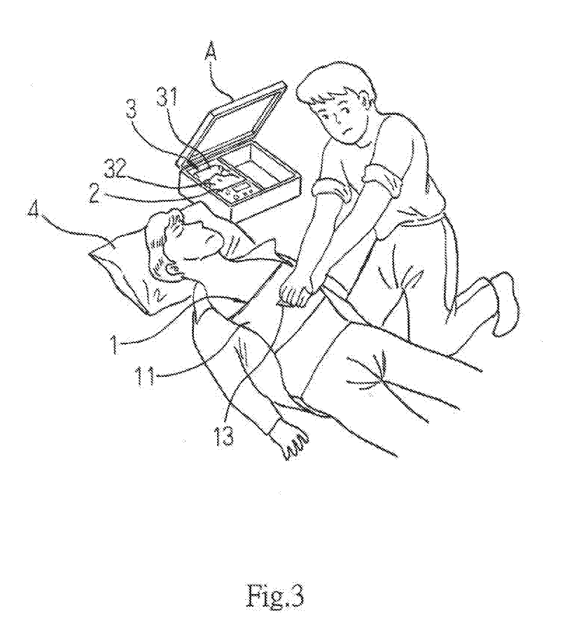

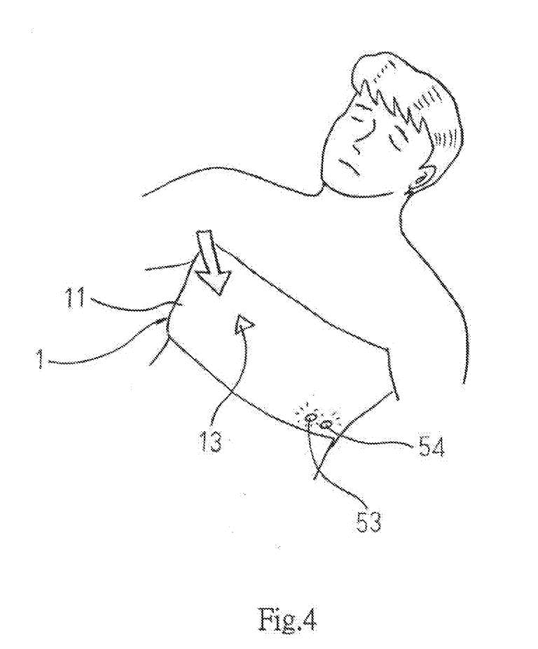

Please refer to both FIGS. 3 and 4, which are drawings of the present invention in use. Please also refer to FIGS. 1 and 2. As the figures show, when using the present invention, take the head rest 4 out of the case body A, place it on the floor or where the patient may lie flat. Place the head of the patient on the head rest 4 after the patient lies flat. Then put the covering member 11 on the chest of the patient, connect the sticky buckles 121 at the tail ends of the two straps 12 to each other to position the covering member 11 and to position the mark 13 at the notch in the center of the chest where the breastbone meets the ribs for assisting the rescuer to administer CPR at a particular position (mark 13) on the covering member 11. According to the standard steps and procedure of CPR, the rescuer kneels beside the patient's shoulder, places the middle finger of one hand on the notch in the center of the patient's chest, and joins the index finger with the middle finger at the bottom rim of the ribs (the particular position on the covering member 11) for positioning. Then the rescuer places the heel of the other hand on the breastbone next to the index finger (a lower half section of the particular position on the covering member 11), put the positioning hand on top of the other hand, and interlock the fingers of both hands or keep them parallel, with the fingers up off the chest wall to prevent them from contacting the ribs. 15 external chest compressions are administered at a rate of 80 to 100 times per minute (with the compression time for 15 compressions totaling around 9 to 11 seconds), and the depth for each compression is around 4 to 5 cm.

During the foregoing CPR administration, the pressure detecting unit 41 disposed on the covering member 11 will constantly detect the compression force and display it on the first display unit 43 to inform the rescuer as to whether the chest compressions are being administered properly. (The standard compression depth is around 4 to 5 cm, which will create a certain amount of pressure. The pressure detecting unit 41 will constantly detect this pressure value. When the compression force is sufficient, the first display unit 43 will blink. If the compression force is insufficient, the first display unit 43 will be off or show steady illumination.)

Further, the heartbeat detecting unit 42 constantly detects the heartbeat signals and displays them on the second display unit 44 to further facilitate proper administration of CPR and enhance rescue efficiency. (Since the heart stops beating during CPR administration, the second display unit 44 is off or shows steady illumination. When the heart starts beating again the second display unit 44 will blink.)

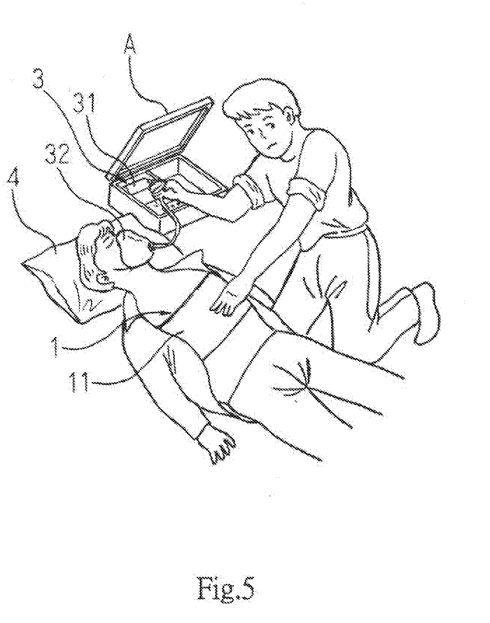

Please refer to FIG. 5, showing a drawing of the present invention providing oxygen. As the figure shows, during the foregoing CPR administration according to the present invention, to provide the patient with a certain amount of oxygen, put the mask 32 on the mouth and nose of the patient and turn on the oxygen bottle 31. The oxygen bottle 31 may provide a proper amount of oxygen to the mask 32 for the patient to breathe in to help the patient recover consciousness.

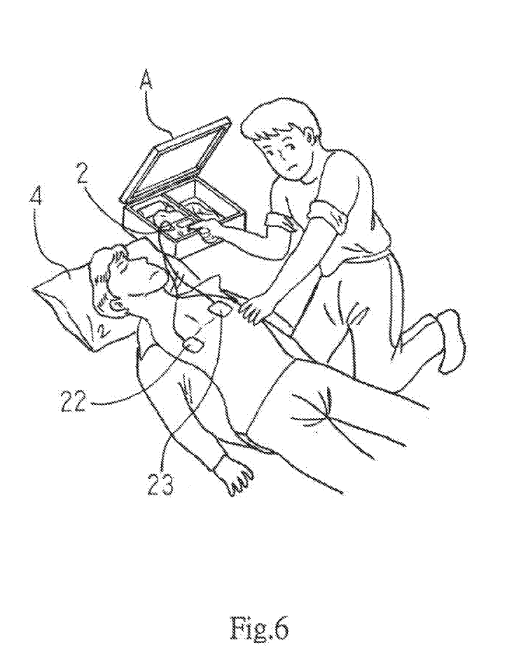

Please refer to FIG. 6, showing a drawing of the AED in use according to the present invention. As the figure shows, after the foregoing CPR administration according to the present invention, if the patient's consciousness is still not recovered, the rescuer may administer an electric shock on the patient. Plug the cables of electrode pads 22 and 23 into the AED 2, place the right electrode pad 22 below the collarbone on the upper right rim of the breastbone, and place the left electrode pad 23 on the outer side of the left nipple with its upper rim at a distance of around 7 cm from the left armpit. After making sure that no one is touching the patient, press the control button 21 to administer an electric shock. After electric shock administration, follow the foregoing steps again and continue CPR administration until the patient recovers consciousness.

The foregoing preferred embodiments of the present invention are illustrated of the present invention rather than limiting of the present invention. It is intended to cover various modifications and changes included within the spirit and scope of the appended claims, the scope of which should be accorded the broadest interpretation so as to encompass all such modifications and similar structures.

In view of the foregoing, the present invention provides a CPR assist device comprising a pressure detecting unit, a heartbeat detecting unit, and a covering member with display units, as well as an AED and an oxygen supplying device. During CPR administration, the present invention may inform the rescuer as to whether chest compressions are being administered properly so that the CPR success rate may be increased.

What is claimed is:

1. A CPR assist device comprising a wearable device, an AED, and an oxygen supplying device, with all the devices stored in a case body, wherein:
on the wearable device are disposed a covering member and straps connecting to the covering member; on the covering member are disposed a pressure detecting unit, a heartbeat detecting unit, a first display unit and a second display unit; in case of a cardiac arrest, the covering member is put on the chest of the patient for the rescuer to administer CPR at a particular position; during CPR administration the pressure detecting unit detects the compression force and displays it on the first display unit to inform the rescuer as to whether the compressions are being administered properly, whereas the heartbeat detecting unit detects the heartbeat signals and the second display unit shows whether the heart has started beating again to facilitate proper CPR administration.

2. A foregoing CPR assist device of claim 1, wherein at the tail ends of the two straps are disposed sticky buckles for the two to connect with each other and for positioning the covering member on the patient's chest.

3. A foregoing CPR assist device of claim 1, wherein the first display unit and the second display unit are light-emitting diodes
